(19) United States
(12) Patent Application Publication
(10) Pub. No.: US 2025/0104815 A1
(43) Pub. Date: Mar. 27, 2025

(54) SEMI-EMPIRICAL WATER MODEL FOR SIMULATING CHEMICAL REACTIONS IN WATER

(71) Applicant: International Business Machines Corporation, Armonk, NY (US)

(72) Inventor: Binquan Luan, Chappaqua, NY (US)

(21) Appl. No.: 18/372,896

(22) Filed: Sep. 26, 2023

(51) Int. Cl. G16C 20/10

(52) U.S. Cl. CPC G16C 20/10

(57) ABSTRACT

A semi-empirical water model based upon an xTB model modified with an energy function (xTB-M) is capable of temperature-dependent predictions of chemical reactions in water. The unmodified xTB model does not provide accurate predictions of chemical reactions in water because the model is incapable of correctly reproducing the caging effect that occurs during chemical reactions in aqueous solutions; thus, chemical reactions simulated with the unmodified model have water density values that are higher than the experimental water density values. The energy function of the xTB-M model corrects the water densities in the unmodified model by accurately calculating the hydrophobic forces between oxygen-pairs in a water simulation thus producing a semi-empirical water model with accurate water density values. By outfitting the water simulation with an NPT or NVT ensemble, the xTB-M model is able to accurately predict the behavior and stability of chemical reactions in water at different temperatures.

### TECHNICAL FIELD

The present invention relates generally to computer modeling of chemical reactions and more specifically, to a semi-empirical model for simulating chemical reactions in water.

### BACKGROUND OF THE INVENTION

To understand the role that water plays in physical and chemical processes, water models must simulate the role of water in various circumstances, including solvation of biomolecules (e.g., proteins and DNAs), surface wetting, interfacial lubrication, water adsorption in porous materials, and nano-fluidics. To date, classical molecular dynamics simulations, first principle-based molecular dynamics simulations, and semi-empirical molecular dynamics simulations have all failed in some way or another to provide platforms for chemical reactions in water. While classical molecular dynamics simulations can successfully reproduce molecular geometry, their inability to model bond forming and breaking makes such simulations unsuitable for modeling chemical reactions in water. First principle molecular dynamics simulations have the capacity to model chemical reactions in water; however, running the calculations to achieve the simulations has been found to be too computationally expensive to be of commercially value. For example, while the enthalpy for a chemical reaction can be readily obtained via density function theory-based calculations, obtaining free energy calculations for chemical reactions in water remains computationally expensive due to the extensive sampling (in the first principle molecular dynamics simulations) that is required for the entropy contribution.

Existing semi-empirical molecular dynamics simulations, such as DFTB, PM6, and xTB, which take into account both theoretical approximations and experimental measurements, fail in their ability to model chemical reactions in water because many of the theoretical predictions required to run the simulations do not align with their experimental counterparts. For example, the semi-empirical xTB method predicts a bulk water density of ˜1450 kg/m³, which is more than 40% higher than experimentally determined bulk water densities measured at room temperature and 1 bar. This high water-density value is the result of stronger-than-normal adhesive interactions among water molecules, which must be corrected in order to yield a reasonable water caging effect for the interacting molecules.

### SUMMARY OF THE INVENTION

The present invention overcomes the need in the art for a more accurate method to simulate chemical reactions in water with a semi-empirical model that has quantum level accuracy for simulating the reactions and is comparable, but 100× faster, than first principle molecular dynamics simulations for the same reactions.

In one embodiment, the present invention relates to a computer program product for simulating a chemical reaction in water, the computer program product comprising one or more computer readable storage media, and program instructions collectively stored on the one or more computer readable storage media, the program instructions comprising: program instructions for computationally building a simulation of at least two reactant molecules in an aqueous solution; program instructions for computationally simulating bonding between the at least two reactant molecules in the aqueous solution with a semi-empirical water model comprising an energy function that calculates hydrophobic forces between pairs of oxygen atoms in the water simulation, wherein the oxygen atoms have pairwise distances (r) in the range of 3.0 Å to 6.2 Å; and program instructions for computationally applying the energy function to the simulation to simulate a chemical reaction of the at least two reactant molecules in the aqueous solution.

In another embodiment, the energy function has a potential well where the oxygen atoms have a pairwise distance of approximately 4.5 Å.

In a further embodiment, the energy function has a potential barrier where the oxygen atoms have a pairwise distance of approximately 5.5 Å.

In another embodiment, the present invention relates to a computer-implemented method comprising: computationally building a simulation of one molecule of each of at least two reactant molecules in an aqueous solution; computationally simulating bonding between the at least two reactant molecules in the aqueous solution with a semi-empirical water model comprising an energy function between pairs of oxygen atoms in the water simulation wherein (i) the oxygen atoms have a pairwise distance in the range of 3.0 Å to 6.2 Å, (ii) the energy function has a potential well where the oxygen atoms have a pairwise distance of approximately 4.5 Å, and (iii) the energy function has a potential barrier where the oxygen atoms have a pairwise distance of approximately 5.5 Å; and computationally applying the energy function to the simulation to simulate a chemical reaction of the at least two reactant molecules in the aqueous solution.

In a further embodiment, the simulation of the at least two reactant molecules in the aqueous solution is a periodic box.

In another embodiment, the periodic box comprises an NPT ensemble specifying a molecular number for the water and reactant molecules in the aqueous solution, a constant pressure for the chemical reaction, and a temperature that is adjusted to simulate the chemical reaction of the at least two reactant molecules in the aqueous solution at different temperatures.

In a further embodiment, the periodic box comprises an NVT ensemble specifying a molecular number for the water and reactant molecules in the aqueous solution, a constant volume for the chemical reaction, and a temperature that is adjusted to simulate the chemical reaction of the at least two reactant molecules in the aqueous solution at different temperatures.

In another embodiment, the present invention relates to a computer-implemented method comprising: computationally building a periodic box of water comprising an NPT or NVT ensemble that includes a constant molecular number for the water in the periodic box and the least two reactant molecules and a temperature for a chemical reaction between the at least two reactant molecules; computationally simulating bonding between the at least two reactant molecules in the periodic water box with a semi-empirical water model comprising an energy function between pairs of oxygen atoms in the periodic water box, wherein the oxygen atoms have pairwise distances in the range of 3.0 Å to 6.2 Å; and computationally applying the energy function to the periodic water box to simulate a chemical reaction of the at least two reactant molecules in water.

In a further embodiment, the semi-empirical water model is an xTB model that is modified with an energy function to form the modified xTB-M model described herein.

In another embodiment, the energy function establishes a water density for the water in the periodic box that aligns with experimental water density values for an aqueous solution comprising the at least two reactant molecules.

Additional aspects and/or embodiments of the invention will be provided, without limitation, in the detailed description of the invention that is set forth below.

### BRIEF DESCRIPTION OF THE DRAWINGS

FIG. 1 is graph showing temperature-dependent water densities in an NPT ensemble as experimentally calculated, as simulated with the conventional xTB (xTB-C) method, and as simulated with the modified xTB (xTB-M) method described herein.

FIG. 2 is a graph showing radial distribution function (RDF) values for a pair of oxygen atoms in a periodic water box obtained experimentally and simulated via a density function theory (DFT), the xTB-C method, and the xTB-M method.

FIG. 3 is a graph showing the potential energy values (kJ/mol) between a pair of oxygen atoms as a function of distance (Å) pursuant to Formulas (1), (2), and (3) as described herein.

FIG. 4 is a diagram of an explicit periodic water box with 229 molecules of water, 1 molecule of monoethanolamine (MEA), and 1 molecule of CO₂.

FIG. 5 is a free energy landscape showing free energy (kcal/mol) as a function of distance (Å) for the chemical reaction between MEA and CO₂ as simulated by the xTB-M method described herein.

FIG. 6 is a free energy landscape showing free energy (kcal/mol) as a function of distance (Å) for the chemical reaction between MEA and CO₂ as simulated by the xTB-C method.

FIG. 7 free energy landscape showing free energy (kcal/mol) as a function of distance (Å) for the chemical reaction between piperazine and CO₂ as simulated by the xTB-M method described herein.

FIG. 8 is a schematic diagram of a computer environment that may be used to implement the semi-empirical xTB-M model described herein.

### DETAILED DESCRIPTION OF THE INVENTION

Set forth below is a description of what are currently believed to be preferred aspects and/or embodiments of the claimed invention. Any alternates or modifications in function, purpose, or structure are intended to be covered by the appended claims. As used in this specification and the appended claims, the singular forms “a,” “an,” and “the” include plural referents unless the context clearly dictates otherwise. The terms “comprise,” “comprised,” “comprises,” and/or “comprising,” as used in the specification and appended claims, specify the presence of the expressly recited components, elements, features, and/or steps, but do not preclude the presence or addition of one or more other components, elements, features, and/or steps.

As used herein the terms “molecular dynamics” and “MD” refer to simulations that describe the interactions of atoms and molecules in a system over a specific period of time through the solving of classical equations of motion. The aim of molecular dynamics simulations is to accurately predict experimental observables of molecular systems.

As used herein, the terms “first principle molecular dynamics,” refers to a simulation technique that combines the sampling power of molecular dynamics simulations with the accuracy of quantum chemical calculations. The first principle meaning that there is no empirical fitting or adjustable parameters.

As used herein, the term “semi-empirical” refers to a practical model that is based upon a combination of measurement and theory.

As used herein, the term “caging effect” refers to how water molecules surround other molecules that are less polar than water in a solution. Water molecules interact with each other through hydrogen bonds. When a non-polar molecule is dropped in a solution of water, hydrogen bonds between the water molecules break to make room for the less-polar molecule (than water); however, due to “hydrophobic forces,” the water molecules may not tightly interact with the less-polar molecular. Hydrophobic forces are the relationship between water and less-polar molecules; when in water, less-polar molecules clump together in order to have minimal contact with the water and in so doing, reduce the entropy barrier for reaction between the less-polar molecules. Water molecules that are distorted by the presence of the less-polar molecule will make new hydrogen bonds and form a cage-like structure around the less-polar molecule.

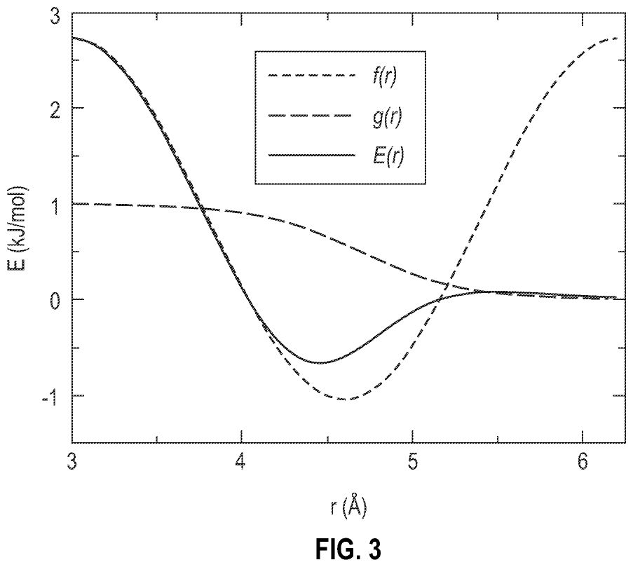

As used herein, the term “potential energy” refers to the stored energy in an object due to its position or state. Once a force has been applied to an object, or an object begins to move, potential energy is converted to kinetic energy (or heat). Within the context of the present invention, a potential energy function (referred to herein as an “energy function”) was introduced between pairs of oxygens atoms in water resulting in the energy function curve (E(r)) shown in FIG. 3.

As used herein, the term “potential energy surface” refers to the potential energy of a collection of atoms in terms of the positions of the atoms where the “surface” defines the potential energy as a function of one or more coordinates. With reference to the energy function curve E(r) of FIG. 3, r represents the interatomic distance of a pair of atoms. In the energy function E(r), E is the height on the energy landscape for all values of r, which gives rise to the potential energy surface.

The term “potential well” refers to energies near a local energy minimum in a potential energy surface. A potential well is shown graphically in FIG. 3 as the area around the lowest energy value.

The term “potential barrier” refers to energies near a local energy maximum in a potential energy surface. A potential barrier is shown graphically in FIG. 3 as the area around the highest energy value.

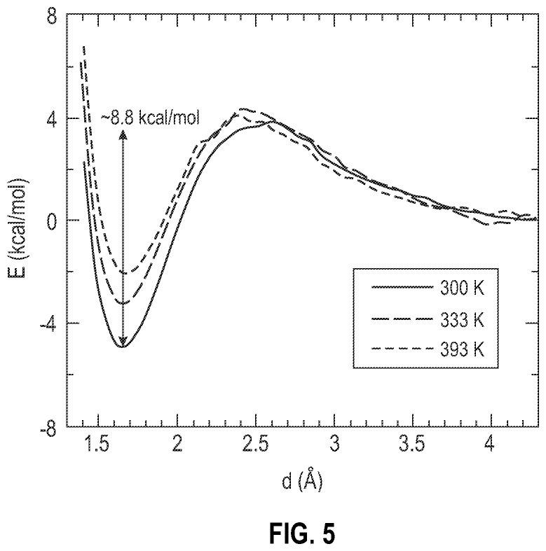

As used herein, the term “free energy” refers to the internal energy of a thermodynamic system that is available to perform work. Free energy is released when bonds break in chemical reactions. Free energy, also known in the art at Gibbs free energy (G), takes into account both enthalpy and entropy. The change in free energy (ΔG) is equal to the subtraction of the enthalpy change minus the product of the temperature and entropy change. As used herein, the term “free energy well,” refers to a region surrounding a local minimum of a free energy surface. Within the context of the present invention, a free energy well occurs when an unbound complex of two molecules in a water cage bind to form a complex (e.g., a zwitterion) and release free energy. Free energy wells are shown graphically in FIGS. 5, 6, and 7 as the area surrounding the lowest point of the curves shown therein.

As used herein, the term “transition state” refers to a region surrounding a local maximum of a free energy surface. Within the context of the present invention, a transition state occurs when two molecules in a periodic box approach each other from a distance and free energies increase before forming a complex of the two unbound molecules surrounded by a water cage. Transition states are shown graphically in FIGS. 5, 6, and 7 as the area surrounding the peaks of the curves shown therein.

As used herein, the term “radial distribution function” or “RDF” refers to a pair correlation function that describes how particles (e.g., atoms, molecules, proteins, celluloids, etc.) are radially packed around each other, i.e., how the density of matter varies as a function of distance from a point.

As used herein, the term “DFT,” which stands for “density-function theory,” refers to a quantum mechanical modeling method that is based on the concept that the energy of a system is a function of its density. DFT simulates the electronic and atomic structures in many-body systems, such as atoms, molecules, condensed phases, and solids.

As used herein, the term “ensemble” refers to a group of systems that are microscopically different, but macroscopically the same. The microscopic state of the group of systems may be the position and/or momentum of atoms, molecules, and/or subatomic particles in the group of systems. The macroscopic state of the group of systems may be one or more of energy, volume, pressure, temperature, and particle number within the group of systems. Examples of ensembles that may be used with the MD simulations described herein are NPT and NVT ensembles.

As used herein, the term “NPT ensemble” refers to a statistical isothermal-isobaric ensemble that is used to characterize the macroscopic state of a closed isothermal-isobaric system with a variable volume. In an NPT ensemble, the particle number (N), pressure (P), and temperature (T) in the group of systems are conserved and for example close to laboratory conditions of ambient temperature and pressure.

As used herein, the term “NVT ensemble” refers to a canonical ensemble that is used to characterize the macroscopic state of a closed system in thermal equilibrium with a heat bath at a fixed temperature. In an NVT ensemble, the particle number (N), volume (V), and temperature (T) are constant while the internal energy of the group of systems is variable.

As used herein, the term “periodic box” refers to a mathematical concept that allows unimpeded motion of particles in a box such that a particle that exits the box through one side reenters it at the opposite size at the same time. Within the context of the present invention, a periodic box is used to simulate chemical reactions in water and in this regard, the term “periodic water box” is also used herein. The conditions within a periodic box will be set by the NPT or NVT ensemble that is used to configure the periodic box. Where the periodic box relates to water, the periodic box may be referred to herein as a “periodic water box.”

As used herein, the term “explicit water” refers to a periodic water box that contains many H₂O molecules (each of which has an all-atom structure) and reproduces bulk water properties in a particular phase. For example, an explicit periodic water box may be designed to measure activation and/or the free energy of a chemical reaction, protein hydration energies, water structure, hydration free energy, etc.

As used herein, the term “xTB” refers to a semi-empirical quantum mechanical method that uses an extended “tight binding” theory to calculate molecular interactions. Tight binding as used in xTB refers to a calculation of electronic band structure based upon superposition of wave functions for isolated atoms located at each atomic site. By way of simple example, a TB molecular model for a frontier atomic orbital containing a single electron is 1,3-butadiene because each carbon atoms contributes one electron to the frontier atomic orbital. More complicated structures, such as ring structures, require matrices that show the interactions between all of the atoms in the molecule.

As used herein, the term “D3 correction” refers to a dispersion correction that is added to a semi-empirical method, such as xTB. The dispersion correction is a function of interatomic distances and contains adjustable parameters that are fitted to compute conformation and energy parameters. A D3 correction is also referred to as a “van der Waals D3 correction” because, as is known to those of skill in the art, van der Waals forces are distant-dependent interactions between atoms or molecules that unlike ionic or covalent bonds, do not result from chemical electronic bonds, but rather, are weak attractions that are susceptible to disturbance and which disappear at longer distances between interacting molecules.

As used herein, the term “Fermi-Dirac distribution function,” refers to a statistical formula for a system of identical fermions (very small particles that are the building blocks of matter (such as electrons) in thermodynamic equilibrium that follow the Pauli exclusion principal, the latter of which provides that two or more identical particles with half-integer spins (i.e., fermions) cannot occupy the same quantum state within a quantum system simultaneously. The Fermi-Dirac distribution function has the following formula:

$$f ( E ) = 1 / {e}^{\frac{E - {E}_{F}}{k T}} + 1$$

where f(E) stands for the probability that a particle will have energy E; E_{F} stands for Fermi energy. For low temperatures, energy states below the Fermi energy have a value of 1 and energy state above the Fermi energy have a value of zero; k is the Boltzmann constant (1.380649×10⁻²³ joules/kelvin); T is a temperature in K; and the +1 is the quantum difference that arises from the fact that the particles are indistinguishable. At absolute zero (T=0), fermions will fill up all available energy states below a level E_{F} (the Fermi energy level). At higher temperatures, some fermions are elevated to levels above the Fermi energy level.

The modified xTB (xTB-M) approach described herein overcomes the problem of simulating chemical reactions in water, which are made difficult due to the hydrophobic forces inherent in water, by superimposing an energy function over existing pair-wise O—O interactions in the conventional xTB (xTB-C) method. The xTB-C model does not provide accurate predictions of chemical reactions in water because the model is incapable of correctly reproducing the caging effect that occurs during chemical reactions in aqueous solutions; thus, chemical reactions simulated with the unmodified model have water density values that are higher than the experimental water density values. The energy function of the xTB-M model corrects the water densities in the unmodified model by accurately calculating the hydrophobic forces between oxygen-pairs in a water simulation thus producing a semi-empirical water model with accurate water density values. The result of the energy function modification is that the simulated RDF for the O—O pairs in water becomes similar to the experimental RDF for the same O—O pair, thus, correcting the inaccurate water density problem that is inherent in the xTB-C model. By outfitting the water simulation with an NPT or NVT ensemble, the xTB-M model is able to accurately predict the behavior and stability of chemical reactions in water at different temperatures. In the following discussion, a water simulation is outfitted with an NPT ensemble; however, it is to be understood that the NPT ensemble is illustrative and that the water simulation could also be outfitted with an NVT ensemble.

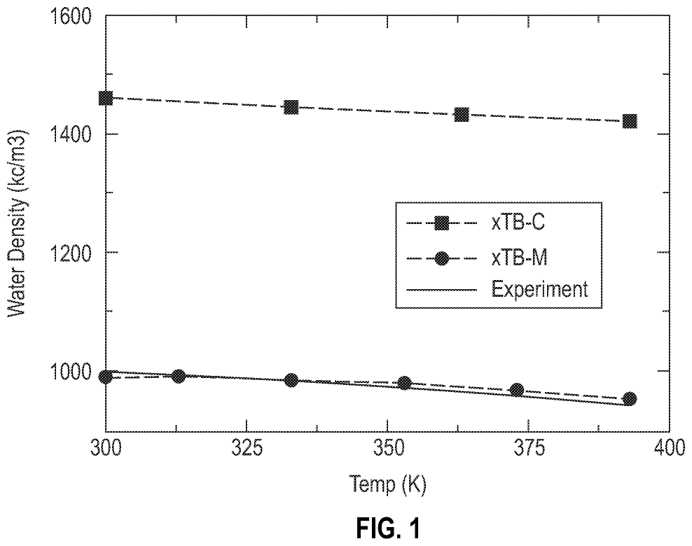

FIG. 1 shows temperature-dependent water densities in an NPT ensemble (N=˜700 atoms; P=1 bar; T=300 K−390K) as experimentally calculated (solid line), as simulated with the xTB-C method (with D3 correction; dashed starred line), and as simulated with the xTB-M method (dashed/dotted line). As shown therein, the simulated water densities with the xTB-C method (˜1450 kg/m³ at 300K) are more than 40% higher than the experimentally calculated values (˜1000 kg/m³ at 300K). The high water density of the xTB-C method results from the simulation calculating stronger than normal adhesive interactions among water molecules, which must be corrected in order to yield the caging effect for the interacting water molecules. The xTB-M model described herein includes such a correction. As shown in FIG. 1, adjusting the xTB-C method to become the xTB-M results in predicted water densities in the range of 300-393K that are comparable to the experimentally calculated water densities. To the best of the inventor's knowledge, the present invention is the first semi-empirical water model that can predict proper temperature-dependent densities of water.

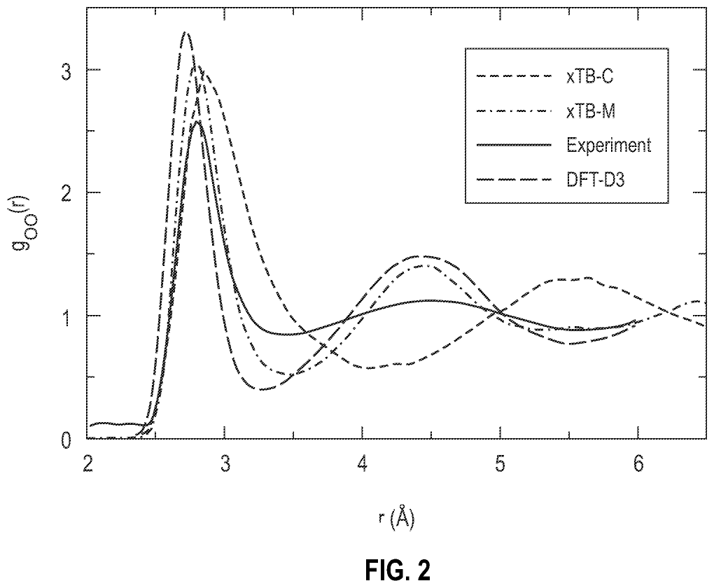

FIG. 2 shows RDF values for O—O pairs in a periodic water box (NPT of N=˜700 atoms; P=1 bar; T=300 K−390K) obtained experimentally, via DFT (with D3 correction), via xTB-C, and via xTB-M. As shown therein, the first peak position for the RDF obtained from the xTB-C method is close to the first experimental peak, but the second peak position for the xTB-C RDF curve deviates significantly from the second RDF experimental peak. More specifically, the positions of the first and second peaks derived from the xTB-C method, which are located at 2.9 Å and 5.6 Å, respectively, are about 0.2 Å and 1.1 Å larger than the corresponding experimentally measured peak positions. Further, while the first peaks from the xTB-C and experimental methods are close in shape, the first peak of the xTB-C method is still much wider than the first experimental peak, suggesting that the water molecules modeled with the xTB-C method are more densely packed and over-coordinated at short distances. Consequently, the position of the first RDF well from the xTB-C method is around 4.2 Å, which is much larger than the 3.5 Å for the first valley of the experimental values.

In contrast to the inaccurate xTB-C RDF results, the xTB-M model accurately predicts the positions of the experimental RDF peaks and valleys. While the predicted RDF peaks and valleys for the xTB-M model are larger and smaller, respectively, than the experimentally determined peaks and valleys, the predicted RDF values from the xTB-M model are significantly better than the predicted RDF values from the xTB-C model, the latter of which shows RDF peaks and wells that do not align with the experimentally determined RDF peaks and wells as discussed above. Although the DFT model showed RDF values to be consistent with the experimental RDF values, the computationally cost of running such as DFT model is significantly greater than the cost associated with running the more efficient xTB-M model.

As discussed above, the xTB-M model comprises an energy function that is superimposed upon the xTB-C model in order to correct the inaccurate O—O RDF values of the xTB-C results for water within a periodic box. The energy function (E) (in kJ/mol) used for the xTB-M model for a pair of oxygen atoms with a distance (r) (in Å) in a periodic box of water is shown mathematically with Formula (1), (2), and (3) and graphically in FIG. 3, wherein represents the distance between a pair of oxygen atoms. In one embodiment, the values for the a, b, c, d and e parameters of Formulas (2) and (3) are listed in Table 1 where the parameters represent optimizations to the xTB-C curve of FIG. 2 to bring the curve closer to the values of the experimental curve for water. In another embodiment, the values for a, b, c (“c” has no units), d, and e in Formulas (2) and (3) are set such that the parameters result in the E(r), f(r), and g(r) curves of FIG. 3.

$$E ( r ) = f ( r ) \cdot g ( r ) , 30 Å \leq r \leq 6.2 Å \qquad ( 1 )$$

$$f ( r ) = {E}_{0} \cdot ( - cos ( π \cdot \frac{r - a}{b} ) + c ) \qquad ( 2 )$$

$$g ( r ) = 1 / 1 + {e}^{\frac{r - d}{e}} \qquad ( 3 )$$

TABLE 1

| Parameters | E₀ | a | b | c | d | e |
|---|---|---|---|---|---|---|
| Values | 1.89 kJ/mol | 4.6 Å | 1.6 Å | 0.45 | 4.7 Å | 0.3 Å |

With reference to Formula (1) and FIG. 3, the E(r) function contains two parts: f(r) and g(r), where 3.0 Å≤r≤6.2 Å, which represent the minimum and maximum experimental r values in FIG. 2. To avoid a sudden force change between a pair of oxygen atoms in an MD simulation, the derivatives of f and g with respect to r at the boundaries (i.e., r=3.0 Å and r=6.2 Å) should be zero. The 3.0 Å≤r≤6.2 Å range represents the range at which the oxygen atoms of water molecules exert forces upon each other while not being involved in chemical bonding. With reference to the E(r) function curve of FIG. 3, the energy function E(r) has an energy well surrounding a local minimum energy potential (E) of ˜4.5 Å and a local maximum energy potential of ˜5.5 Å. In one embodiment, the energy function E(r) is established by using the parameters of Table 1. In another embodiment, the energy function is established by using parameters that are similar to, but not identical to the parameters of Table 1. In a further embodiment, the energy function is established by using parameters that surround the parameters of Table 1. In another embodiment, the energy function is established by applying parameters that yield an E(r) curve as shown in FIG. 3.

With reference to Formula (2) and FIG. 3, the f(r) function contains a cosine function that specifies an energy well with a minimum at r=a, a half-width of b, and an energy-shift of c×E₀. To ensure that the RDF for the O—O pair has its second peak located near the 4.6 Å experimental value shown in FIG. 2, the value of a is set at 4.6 Å. With the value of b set to be 1.6 Å (one-half the 3.2 Å width of the range 3.0 Å≤r≤6.2 Å), as shown in the f(r) function curve of FIG. 3, f(r) has its maximum energy potentials (E) at 3.0 Å and 6.2 Å, which shifts the position of the first xTB-C peak in FIG. 2 from 2.9 Å to a smaller value that is close to the experimental value (i.e., a value that becomes the xTB-M value for the first curve in FIG. 2).

With reference to Formula (3), g(r) is similar to the Fermi-Dirac distribution function at a finite temperature for fermions, which gradually switches off interactions at larger distances, as is shown in the g(r) function curve of FIG. 3.

In one embodiment, the present invention comprises building a simulation of at least two reactant molecules in an aqueous solution; simulating bonding between the at least two reactant molecules in the aqueous solution with a semi-empirical water model modified by an energy function that is dependent upon oxygen atoms in the aqueous solution have pairwise distances in the range of 3.0 Å to 6.2 Å; and applying the energy function to the simulation to simulate a chemical reaction of the at least two reactant molecules in the aqueous solution.

In another embodiment, the present invention comprises building a simulation of one molecule of each of at least two reactant molecules in an aqueous solution; simulating bonding between the at least two reactant molecules in the aqueous solution with a semi-empirical water model modified by an energy function that is dependent upon oxygen atoms in the aqueous solution having pairwise distances in the range of 3.0 Å to 6.2 Å, wherein the energy function has a potential well when the oxygen atoms are at a pairwise distance of ˜4.5 Å and a potential barrier when the oxygen atoms are at a pairwise distance of ˜5.5 Å; and applying the energy function to the simulation to simulate a chemical reaction of the at least two reactant molecules in the aqueous solution.

In a further embodiment, the present invention comprises building a periodic box of water comprising an NPT or NVT ensemble that includes a constant molecular number for the water in the periodic box and the least two reactant molecules and a temperature for a chemical reaction between the at least two reactant molecules; computationally simulating bonding between the at least two reactant molecules in the periodic water box with a semi-empirical water model modified with an energy function that is dependent upon oxygen atoms in the periodic water box having pairwise distances in the range of 3.0 Å to 6.2 Å; and applying the energy function to the periodic water box to simulate a chemical reaction of the at least two reactant molecules in water.

In another embodiment, the semi-empirical water model is an xTB model.

In a further embodiment, the energy function calculates a water density for the water in the periodic box that aligns with experimental water density values for an aqueous solution comprising the at least two reactant molecules.

In another embodiment, the NPT or NVT ensemble is adjusted with different temperatures to simulate the chemical reaction of the at least two reactant molecules in the aqueous solution at different temperatures.

As proof of concept, the xTB-M method described herein was used to model CO₂ capture by amine-based products via zwitterion formation resulting from CO₂-amine interactions in water, where the zwitterion represents an intermediate state before a final carbamate reaction product. While the reaction between an amine group and a CO₂ molecule in the gas phase is energetically prohibited, in the presence of water, a free energy well occurs such that zwitterion formation becomes energetically favorable. Formulas (4) and (5) show the general two-step reaction mechanism involving an intermediate CO₂-amine zwitterion, where X represents a generalization of a basic molecule, such as OH⁻, H₂O, or the solvents MEA (monoethanolamine) (Example 1) or piperazine (Example 2):

RNH₂+CO₂RN⁺H₂COO⁻ (4)

RN⁺H₂COO⁻X RNHCOO⁻XH⁺ (5)

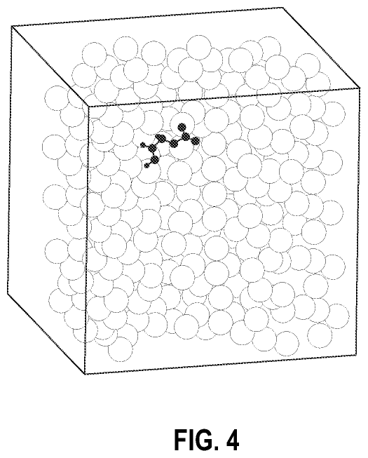

FIG. 4 shows a simulated explicit water periodic box with an NPT ensemble of 229 water molecules, one molecule of MEA, and one molecule of CO₂, which is used for MD simulations of CO₂ capture by MEA in water.

FIG. 5 shows the free energy landscape for the xTB-M simulation of CO₂ capture by MEA in the explicit periodic water box of FIG. 4 at 1 bar and three different temperatures: 300 K, 333K, and 393K (Example 1). Referring first to the simulation at 300K, when CO₂ and MEA approach each other from a distance of ˜4.3 Å, free energies increase by about 4 kcal/mol and peak at ˜2.5 Å, indicating a transition state for the reaction; specifically, the formation of an unbound MEA-CO₂ complex surrounded by a water cage. When even closer to each other, free energies decrease and exhibit a minimum, i.e., a free energy well, at ˜1.65 Å, at which point the CO₂ and MEA chemically bond to form a zwitterion, which is subsequently deprotonated to become a carbamate. The activation free energy for the CO₂ release (measured from the energy well of the zwitterion to the peak of the transition state) is ˜8.8 kcal/mol at 300K. At 333K and 393K, the xTB-M method predicts an activation free energy for the reaction of CO₂ and MEA of 7.6 kcal/mol and 6.1 kcal/mol, respectively. All of the free energy predictions from the xTB-M method fall within the 6˜10 kcal/mol measurements obtained with the more computationally expensive DFT model.

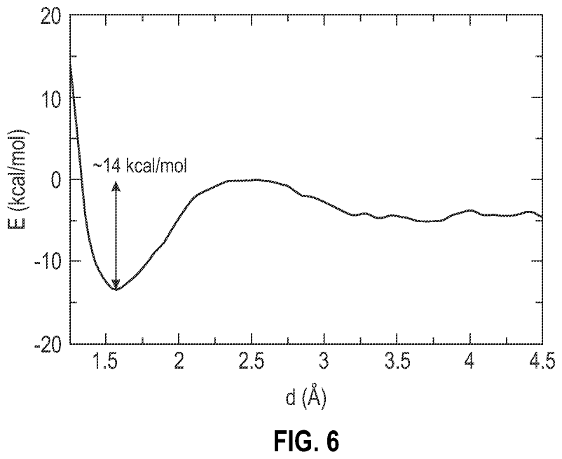

By way of comparison, FIG. 6 shows the free energy landscape for the xTB-C simulation of CO₂ capture by MEA in the explicit periodic water box of FIG. 4 at 1 bar and 300K. Under this xTB-C simulation, the activation free energy of the CO₂ and MEA measured from the local maximum of the transition state (the transition state peak) to the local minimum of the energy well is ˜14 kcal/mol, which is significantly higher than both the 8.8 kcal/mol xTB-M value and 6˜10 kcal/mol DFT values. The high activation free-energy value for the xTB-C method is due to the incorrectly high water density that is built into the xTB-C method as previously discussed.

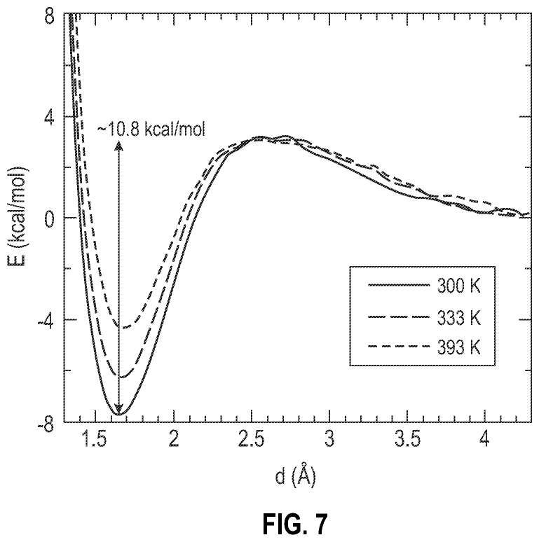

As another example, FIG. 7 shows the free energy landscape for the xTB-M simulation of CO₂ capture by piperazine in an explicit periodic water box with 229 water molecules, one molecule of CO₂ and one molecule of piperazine at 1 bar and three different temperatures: 300K, 333K, and 393K (Example 2). Referring first to the simulation at 300K, when the two molecules approach each other from a distance of ˜4.3 Å, the free energies increase by about 3 kcal/mol to a transition state peak of ˜2.6 Å. As the two molecules continue to approach each other, the free energy well appears at ˜1.65 Å at which point the CO₂ and piperazine chemically bond to form a zwitterion, which is subsequently deprotonated. The activation free energy for the reaction of the CO₂ and piperazine is ˜10.8 kcal/mol at 300K. At 333K and 393K, the xTB-M method predicts activation free energies for the reaction of CO₂ and piperazine of 9.4 kcal/mol and 7.4 kcal/mol, respectively. The deeper well and lower transition state barrier in the free energy landscape for the piperazine-CO₂ complex versus the MEA-CO₂ complex suggests that the formation of the CO₂-piperadine zwitterion is more stable than the CO₂-MEA zwitterion.

Within the context of CO₂ capture, given the large number of primary, secondary and tertiary amines in existence the xTB-M method described herein provides a fast-screening method for amine-based CO₂ capture. It is to be understood that while application of the xTB-M model is described herein for CO₂ capture by amine-based molecules, the semi-empirical xTB-M model has application for the computational study of any chemical reaction in water.

Various aspects of the present disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

Examples of software that may be used to build a periodic box include any software package with DFT-based calculations. Examples of open-source DFT software packages include, without limitation, ABINIT, ACES, ADF, BerkeleyGW, BigDFT, COLUMBUS, CONQUEST, CP2K, Dalton, DFTK, DIRAC, FLEUR, FreeON, GPAW, MADNESS, MOPAC, MPQC, NWChem, Octopus, OpenMx, PARSEC, PSI, PyQuante, PySCF, Qbox, Quantum ESPRESSO, SAMSON, SIESTA, and Yambo. Additional software packages that may be used to build a periodic box include, without limitation, Hyperchem, FixBox, NAMD, and PACKMOL Software to run the conventional xTB model (xTB-C) is available at xtb-docs.com and may be modified to the xTB-M model using the energy function as described herein.

A computer program product embodiment (“CPP embodiment” or “CPP”) is a term used in the present disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include: diskette, hard disk, random access memory (RAM), read-only memory (ROM), erasable programmable read-only memory (EPROM or Flash memory), static random-access memory (SRAM), compact disc read-only memory (CD-ROM), digital versatile disk (DVD), memory stick, floppy disk, mechanically encoded device (such as punch cards or pits/lands formed in a major surface of a disc) or any suitable combination of the foregoing. A computer readable storage medium, as that term is used in the present disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation or garbage collection, but this does not render the storage device as transitory because the data is not transitory while it is stored.

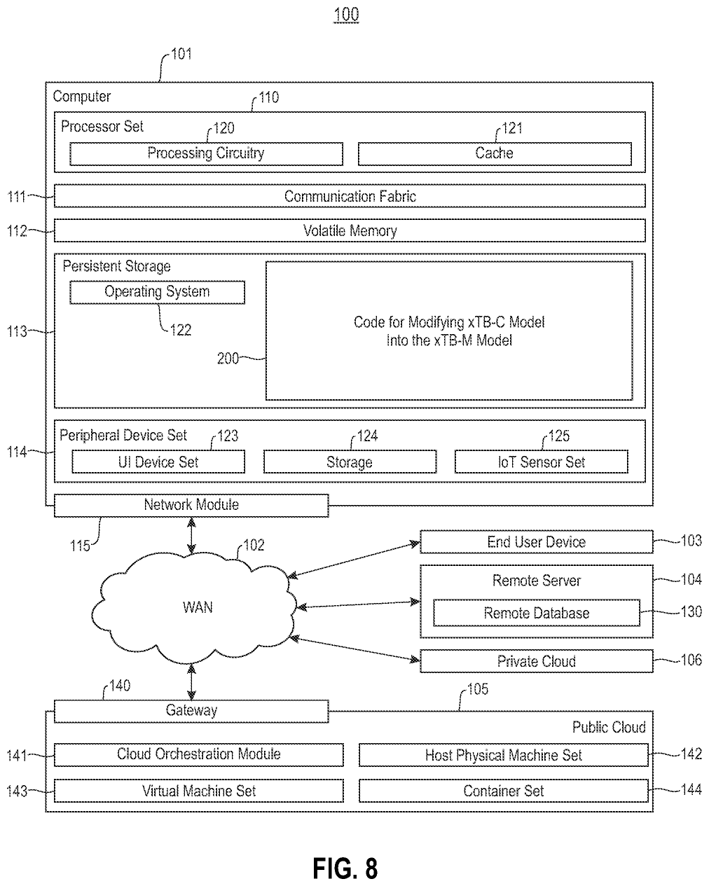

With reference to FIG. 8, a computing environment 100 contains an example of an environment for the execution of at least some of the computer code involved in performing the inventive methods, such as code for modifying an xTB-C model into the xTB-M model described herein 200. In addition to block 200, computing environment 100 includes, for example, computer 101, wide area network (WAN) 102, end user device (EUD) 103, remote server 104, public cloud 105, and private cloud 106. In this embodiment, computer 101 includes processor set 110 (including processing circuitry 120 and cache 121), communication fabric 111, volatile memory 112, persistent storage 113 (including operating system 122 and block 200, as identified above), peripheral device set 114 (including a user interface (UI) device set 123, storage 124, and Internet of Things (IoT) sensor set 125), and network module 115. Remote server 104 includes remote database 130. Public cloud 105 includes gateway 140, cloud orchestration module 141, host physical machine set 142, virtual machine set 143, and container set 144.

COMPUTER 101 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network or querying a database, such as remote database 130. As is well understood in the art of computer technology, and depending upon the technology, performance of a computer-implemented method may be distributed among multiple computers and/or between multiple locations. On the other hand, in this presentation of computing environment 100, detailed discussion is focused on a single computer, specifically computer 101, to keep the presentation as simple as possible. Computer 101 may be located in a cloud, even though it is not shown in a cloud in FIG. 8. On the other hand, computer 101 is not required to be in a cloud except to any extent as may be affirmatively indicated.

PROCESSOR SET 110 includes one, or more, computer processors of any type now known or to be developed in the future. Processing circuitry 120 may be distributed over multiple packages, for example, multiple, coordinated integrated circuit chips. Processing circuitry 120 may implement multiple processor threads and/or multiple processor cores. Cache 121 is memory that is located in the processor chip package(s) and is typically used for data or code that should be available for rapid access by the threads or cores running on processor set 110. Cache memories are typically organized into multiple levels depending upon relative proximity to the processing circuitry. Alternatively, some, or all, of the cache for the processor set may be located “off chip.” In some computing environments, processor set 110 may be designed for working with qubits and performing quantum computing.

Computer readable program instructions are typically loaded onto computer 101 to cause a series of operational steps to be performed by processor set 110 of computer 101 and thereby effect a computer-implemented method, such that the instructions thus executed will instantiate the methods specified in flowcharts and/or narrative descriptions of computer-implemented methods included in this document (collectively referred to as “the inventive methods”). These computer readable program instructions are stored in various types of computer readable storage media, such as cache 121 and the other storage media discussed below. The program instructions, and associated data, are accessed by processor set 110 to control and direct performance of the inventive methods. In computing environment 100, at least some of the instructions for performing the inventive methods may be stored in block 200 in persistent storage 113.

COMMUNICATION FABRIC 111 is the signal conduction path that allows the various components of computer 101 to communicate with each other. Typically, this fabric is made of switches and electrically conductive paths, such as the switches and electrically conductive paths that make up busses, bridges, physical input/output ports and the like. Other types of signal communication paths may be used, such as fiber optic communication paths and/or wireless communication paths.

VOLATILE MEMORY 112 is any type of volatile memory now known or to be developed in the future. Examples include dynamic type random access memory (RAM) or static type RAM. Typically, volatile memory 112 is characterized by random access, but this is not required unless affirmatively indicated. In computer 101, the volatile memory 112 is located in a single package and is internal to computer 101, but, alternatively or additionally, the volatile memory may be distributed over multiple packages and/or located externally with respect to computer 101.

PERSISTENT STORAGE 113 is any form of non-volatile storage for computers that is now known or to be developed in the future. The non-volatility of this storage means that the stored data is maintained regardless of whether power is being supplied to computer 101 and/or directly to persistent storage 113. Persistent storage 113 may be a read only memory (ROM), but typically at least a portion of the persistent storage allows writing of data, deletion of data and re-writing of data. Some familiar forms of persistent storage include magnetic disks and solid-state storage devices. Operating system 122 may take several forms, such as various known proprietary operating systems or open-source Portable Operating System Interface-type operating systems that employ a kernel. The code included in block 200 typically includes at least some of the computer code involved in performing the inventive methods.

PERIPHERAL DEVICE SET 114 includes the set of peripheral devices of computer 101. Data communication connections between the peripheral devices and the other components of computer 101 may be implemented in various ways, such as Bluetooth connections, Near-Field Communication (NFC) connections, connections made by cables (such as universal serial bus (USB) type cables), insertion-type connections (for example, secure digital (SD) card), connections made through local area communication networks and even connections made through wide area networks such as the internet. In various embodiments, UI device set 123 may include components such as a display screen, speaker, microphone, wearable devices (such as goggles and smart watches), keyboard, mouse, printer, touchpad, game controllers, and haptic devices. Storage 124 is external storage, such as an external hard drive, or insertable storage, such as an SD card. Storage 124 may be persistent and/or volatile. In some embodiments, storage 124 may take the form of a quantum computing storage device for storing data in the form of qubits. In embodiments where computer 101 is required to have a large amount of storage (for example, where computer 101 locally stores and manages a large database) then this storage may be provided by peripheral storage devices designed for storing very large amounts of data, such as a storage area network (SAN) that is shared by multiple, geographically distributed computers. IoT sensor set 125 is made up of sensors that can be used in Internet of Things applications. For example, one sensor may be a thermometer and another sensor may be a motion detector.

NETWORK MODULE 115 is the collection of computer software, hardware, and firmware that allows computer 101 to communicate with other computers through WAN 102. Network module 115 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 115 are performed on the same physical hardware device. In other embodiments (for example, embodiments that utilize software-defined networking (SDN)), the control functions and the forwarding functions of network module 115 are performed on physically separate devices, such that the control functions manage several different network hardware devices. Computer readable program instructions for performing the inventive methods can typically be downloaded to computer 101 from an external computer or external storage device through a network adapter card or network interface included in network module 115.

WAN 102 is any wide area network (for example, the internet) capable of communicating computer data over non-local distances by any technology for communicating computer data, now known or to be developed in the future. In some embodiments, the WAN 102 may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and edge servers.

END USER DEVICE (EUD) 103 is any computer system that is used and controlled by an end user (for example, a customer of an enterprise that operates computer 101), and may take any of the forms discussed above in connection with computer 101. EUD 103 typically receives helpful and useful data from the operations of computer 101. For example, in a hypothetical case where computer 101 is designed to provide a recommendation to an end user, this recommendation would typically be communicated from network module 115 of computer 101 through WAN 102 to EUD 103. In this way, EUD 103 can display, or otherwise present, the recommendation to an end user. In some embodiments, EUD 103 may be a client device, such as thin client, heavy client, mainframe computer, desktop computer and so on.

REMOTE SERVER 104 is any computer system that serves at least some data and/or functionality to computer 101. Remote server 104 may be controlled and used by the same entity that operates computer 101. Remote server 104 represents the machine(s) that collect and store helpful and useful data for use by other computers, such as computer 101. For example, in a hypothetical case where computer 101 is designed and programmed to provide a recommendation based on historical data, then this historical data may be provided to computer 101 from remote database 130 of remote server 104.

PUBLIC CLOUD 105 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economies of scale. The direct and active management of the computing resources of public cloud 105 is performed by the computer hardware and/or software of cloud orchestration module 141. The computing resources provided by public cloud 105 are typically implemented by virtual computing environments that run on various computers making up the computers of host physical machine set 142, which is the universe of physical computers in and/or available to public cloud 105. The virtual computing environments (VCEs) typically take the form of virtual machines from virtual machine set 143 and/or containers from container set 144. It is understood that these VCEs may be stored as images and may be transferred among and between the various physical machine hosts, either as images or after instantiation of the VCE. Cloud orchestration module 141 manages the transfer and storage of images, deploys new instantiations of VCEs and manages active instantiations of VCE deployments. Gateway 140 is the collection of computer software, hardware, and firmware that allows public cloud 105 to communicate through WAN 102.

Some further explanation of virtualized computing environments (VCEs) will now be provided. VCEs can be stored as “images.” A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

PRIVATE CLOUD 106 is similar to public cloud 105, except that the computing resources are only available for use by a single enterprise. While private cloud 106 is depicted as being in communication with WAN 102, in other embodiments a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment, public cloud 105 and private cloud 106 are both part of a larger hybrid cloud.

The descriptions of the various aspects and/or embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the aspects and/or embodiments, the practical application or technical improvement over technologies found in the marketplace, or to enable others of ordinary skill in the art to understand the aspects and/or embodiments disclosed herein.

### EXPERIMENTAL

The following examples are set forth to provide those of ordinary skill in the art with a complete disclosure of how to make and use the aspects and embodiments of the invention as set forth herein. While efforts have been made to ensure accuracy with respect to variables such as amounts, temperature, etc., experimental error and deviations should be considered. Unless indicated otherwise, parts are parts by weight, temperature is degrees centigrade, and pressure is at or near atmospheric. All components were obtained commercially unless otherwise indicated.

In the following examples, the CP2K open-source molecular dynamics software package (available at cp2k.org) with a Perdew-Burke-Ernzerhof (PBE) function was used in first-principle calculations for a periodic box of explicit water containing an NPT ensemble at three different temperatures. Water densities at the different temperatures were maintained using a velocity-rescaling thermostat. The well-tempered MD algorithm (Barducci et al., Phys. Rev. Lett. 100(2):020603, pp. 1˜4 (2018)) was used for smooth convergence. The graph.popt code in the CP2K software package was used to construct free energy landscapes from the simulation output.

### Example 1

### Application of the Semi-Empirical Xtb-M Method to Predict Co₂ Capture by an MEA Solvent in a Periodic Water Box

A periodic water box (hereinafter “explicit water model”) with an NPT ensemble of N=229 water molecules and P=1 bar was constructed for three different temperatures: T=300, 333, and 393 K. One molecule of monoethanolamine (MEA) and one molecule of CO₂ was added to the periodic box to form an explicit water model. The xTB-M method described herein was used to simulate the chemical reaction of the MEA and CO₂ in the explicit water model at each temperature. Each xTB-M simulation was run at 500 picosecond (10⁻¹² s) with a time step of 1 femtosecond (10⁻¹⁵ sec) for the amine-CO₂ complex formation in the explicit water. The N—C distance within the amine-CO₂ complex (with the nitrogen from the amine and the C from the CO₂) was used as a collective variable for CO₂ chemisorption/release in the MD simulation. Free energy landscapes for zwitterion formation resulting from the chemical reaction between the MEA and CO₂ were constructed for the three different temperatures (FIG. 5).

### Example 2

### Application of the Semi-Empirical XTB-M Method to Predict Co₂ Capture by a Piperazine Solvent in a Periodic Water Box

A periodic water box (hereinafter “explicit water model”) with an NPT ensemble of N=229 water molecules and P=1 bar was constructed for three different temperatures: T=300, 333, and 393 K. One molecule of piperazine and one molecule of CO₂ was added to the periodic water box to form an explicit water model. The xTB-M method described herein was used to simulate the chemical reaction of the piperazine and CO₂ in the explicit water model at each temperature. Each xTB-M simulation was run at 500 picosecond (10⁻¹² sec) with a time step of 1 femtosecond (10⁻¹⁵) for the amine-CO₂ complex formation in the explicit water. The N—C distance within the amine-CO₂ complex (with the nitrogen from the amine and the C from the CO₂) was used as a collective variable for CO₂ chemisorption/release in the MD simulation. Free energy landscapes for zwitterion formation resulting from the chemical reaction between the piperazine and CO₂ were constructed for the three different temperatures (FIG. 7).

I claim:

1. A computer program product for simulating a chemical reaction in water, the computer program product comprising one or more computer readable storage media, and program instructions collectively stored on the one or more computer readable storage media, the program instructions comprising:
program instructions for computationally building a simulation of at least two reactant molecules in an aqueous solution;
program instructions for computationally simulating bonding between the at least two reactant molecules in the aqueous solution with a semi-empirical water model comprising an energy function that calculates hydrophobic forces between pairs of oxygen atoms in the water simulation, wherein the oxygen atoms have pairwise distances (r) in the range of 3.0 Å to 6.2 Å; and
program instructions for computationally applying the energy function to the simulation to simulate a chemical reaction of the at least two reactant molecules in the aqueous solution.

2. The computer program product of claim 1, wherein the energy function has a potential well where the oxygen atoms have a pairwise distance of approximately 4.5 Å.

3. The computer program product of claim 1, wherein the energy function has a potential barrier where the oxygen atoms have a pairwise distance of approximately 5.5 Å.

4. The computer program product of claim 1, wherein the semi-empirical water model is an xTB model.

5. The computer program product of claim 1, wherein the simulation of the at least two reactant molecules in the aqueous solution is a periodic box.

6. The computer program product of claim 5, wherein the energy function establishes a water density for the water in the periodic box that aligns with experimental water density values for an aqueous solution comprising the at least two reactant molecules.

7. The computer program product of claim 5, wherein the periodic box comprises an NPT ensemble specifying a molecular number for the water and reactant molecules in the aqueous solution, a constant pressure for the chemical reaction, and a temperature that is adjusted to simulate the chemical reaction of the at least two reactant molecules in the aqueous solution at different temperatures.

8. The computer program product of claim 5, wherein the periodic box comprises an NVT ensemble specifying a molecular number for the water and reactant molecules in the aqueous solution, a constant volume for the chemical reaction, and a temperature that is adjusted to simulate the chemical reaction of the at least two reactant molecules in the aqueous solution at different temperatures.

9. A computer-implemented method comprising:
computationally building a simulation of one molecule of each of at least two reactant molecules in an aqueous solution;
computationally simulating bonding between the at least two reactant molecules in the aqueous solution with a semi-empirical water model comprising an energy function between pairs of oxygen atoms in the water simulation wherein (i) the oxygen atoms have a pairwise distance in the range of 3.0 Å to 6.2 Å, (ii) the energy function has a potential well where the oxygen atoms have a pairwise distance of approximately 4.5 Å, and (iii) the energy function has a potential barrier where the oxygen atoms have a pairwise distance of approximately 5.5 Å; and
computationally applying the energy function to the simulation to simulate a chemical reaction of the at least two reactant molecules in the aqueous solution.

10. The computer-implemented method of claim 9, wherein the semi-empirical water model is an xTB model.

11. The computer-implemented method of claim 9, wherein the simulation of the at least two reactant molecules in the aqueous solution is a periodic water box.

12. The computer-implemented method of claim 11, wherein the energy function establishes a water density for the water in the periodic water box that aligns with experimental water density values for an aqueous solution comprising the at least two reactant molecules.

13. The computer-implemented method of claim 11, wherein the periodic water box comprises an NPT ensemble specifying a molecular number for the water and reactant molecules in the aqueous solution, a constant pressure for the chemical reaction, and a temperature that is adjusted to simulate the chemical reaction of the at least two reactant molecules in the aqueous solution at different temperatures.

14. The computer-implemented method of claim 11, wherein the periodic box comprises an NVT ensemble specifying a molecular number for the water and reactant molecules in the aqueous solution, a constant volume for the chemical reaction, and a temperature that is adjusted to simulate the chemical reaction of the at least two reactant molecules in the aqueous solution at different temperatures.

15. A computer-implemented method comprising:
computationally building a periodic box of water comprising an NPT or NVT ensemble that includes a constant molecular number for the water in the periodic box and the least two reactant molecules and a temperature for a chemical reaction between the at least two reactant molecules;
computationally simulating bonding between the at least two reactant molecules in the periodic water box with a semi-empirical water model comprising an energy function between pairs of oxygen atoms in the periodic water box, wherein the oxygen atoms have pairwise distances in the range of 3.0 Å to 6.2 Å; and
computationally applying the energy function to the periodic water box to simulate a chemical reaction of the at least two reactant molecules in water.

16. The computer-implemented method of claim 15, wherein the energy function has an potential well where the oxygen atoms have a pairwise distance of approximately 4.5 Å.

17. The computer-implemented method of claim 15, wherein the energy function has a potential barrier where the oxygen atoms have a pairwise distance of approximately 5.5 Å.

18. The computer-implemented method of claim 15, wherein the semi-empirical water model is an xTB model.

19. The computer-implemented method of claim 15, wherein the energy function establishes a water density for the water in the periodic box that aligns with experimental water density values for an aqueous solution comprising the at least two reactant molecules.

20. The computer-implemented method of claim 15, wherein the temperature of the NPT or NVT ensemble is adjusted to simulate the chemical reaction of the at least two reactant molecules in water at different temperatures.
